(19) United States
(12) Patent Application Publication
(10) Pub. No.: US 2022/0126131 A1
(43) Pub. Date: Apr. 28, 2022

(54) SANITIZING DEVICE FOR FACE MASKS

(71) Applicant: Donald SPECTOR, Jupiter, FL (US)

(72) Inventor: Donald SPECTOR, Jupiter, FL (US)

(21) Appl. No.: 17/097,132

(22) Filed: Nov. 13, 2020

Related U.S. Application Data
(60) Provisional application No. 63/105,248, filed on Oct. 24, 2020.

(51) Int. Cl. A62B 23/02; A61L 9/20

(52) U.S. Cl. CPC A62B 23/025; A61L 2209/12; A61L 9/20

(57) ABSTRACT

A sanitizing device has a housing having a central aperture and an ultraviolet (UV) light-emitting diode (LED) assembly mounted in the housing. The UV LED assembly is formed of a plurality of connected UV LEDs arranged circumferentially around the central aperture so as to project UV light into the aperture. A power source is connected to the UV LED assembly to supply power to the assembly. The housing is configured with overhanging front and rear faces, so that the UV LED assembly is recessed within the housing and is not visible when viewing the housing directly from the front or rear. This arrangement protects the user and others from excessive UV exposure during use. The sanitizing device is configured for attachment to a face mask so that the inhalations and exhalations of the wearer pass through the sanitizing device when the mask is worn.

### CROSS-REFERENCE TO RELATED APPLICATIONS

This application claims priority under 35 USC 119(e) of U.S. Provisional Patent Application No. 63/105,248, filed on Oct. 24, 2020, the disclosure of which is herein incorporated by reference.

### BACKGROUND OF THE INVENTION

### 1. Field of the Invention

The present invention relates to a sanitizing device for attachment to a face mask and which utilizes ultraviolet light to sterilize the wearer's exhalations as they are emitted from the mask.

### 2. The Prior Art

The respiratory system represents a closed air system with a self-contained cavity, much smaller than a car cabin, or room. Unlike man-made rooms or vehicles in which air ducts and fans are used to circulate air, the human body uses inhalation and expiration as a mechanism to fill the respiratory system, thereby providing needed oxygen to the blood supply.

The respiratory system is also a pathway into the body for viruses, bacteria and other harmful pathogens. Inhalation can bring such organisms into the person and exhalation can deliver particles containing such organisms to others, thereby spreading disease.

Face masks are often worn by medical professionals to protect patents from any viruses or bacteria that may be harbored by the wearer. The face masks also provide a degree of protection to the wearer from outside sources. These masks can be made of a variety of materials, such as paper, fabric or various nonwoven materials. While these masks can be somewhat effective in preventing the virus particles from escaping through the mask, their efficacy is limited due to an imprecise fit and limited filtration. it would be desirable to equip the masks with a way to kill the virus particles so that even those that escape from the mask cannot infect the public.

### SUMMARY OF THE INVENTION

This object is accomplished according to the invention by a sanitizing device comprising a housing having a central aperture and an ultraviolet (UV) light-emitting diode (LED) assembly mounted in the housing. The UV LED assembly is formed of a plurality of connected UV LEDs arranged circumferentially around the central aperture so as to project UV-C light into the aperture. UV-C light is a short-wave ultraviolet light that has been found to be effective in killing viruses in the air and on surfaces. Far UV-C light (e.g. 222 nm wavelength) is preferably used, as exposure to it has less adverse effects on humans. A power source is connected to the UV LED assembly to supply power to the assembly. The housing is configured with overhanging front and rear faces, so that the UV LED assembly is recessed within the housing and is not visible when viewing the housing directly from the front or rear. This arrangement protects the user and others from excessive UV exposure during use.

There is an attachment layer connected to a rear surface of the housing. The attachment layer is configured for attaching the sanitizing device to a face mask. The attachment layer can be any suitable attachment means, such as a releasable adhesive or a hook-and-loop type fastener, that follows the toroidal shape of the housing. If a hook-and-loop type fastener is used, one side of the fastener is affixed to the housing, and then a plurality of corresponding layers of the other side of the fastener are provided for attachment to a face mask, as new fastener is required each time the mask is discarded an a new one is used. Each side of the fastener can be affixed to the housing or the mask via an adhesive substance. The power source also has an attachment layer connected to its back surface. The attachment layer can also be any suitable attachment means, such as an adhesive or hook-and-loop type closure. A switch can be connected to the housing to turn the LEDs on and off. An indicator light can be connected to the switch so that the user, who may not be able to see the LEDs that are recessed in the housing, will know whether the LEDs are on or off.

To further prevent exposure by the LEDs, a filter cover can be placed over the aperture in the housing, on the front and/or rear sides of the housing.

The power source can be any suitable source of electrical power for the LEDs. In one embodiment, the power source is a battery. The battery is preferably configured to be as small and as flat as possible, so that it can be easily adhered to a face mask. The battery is connected to the LED assembly via a wire.

The sanitizing device according to the invention is ideally used on disposable paper face masks to sanitize the user's inhalations and exhalations. The housing is affixed to a front or rear central portion of the face mask, adjacent the wearer's mouth, so as to capture the majority of air exhaled by the wearer. The battery can be affixed to any suitable location on the mask. Once the mask has been used, the sanitizing device can be removed from the mask and re-used on a new mask by affixing the housing and battery to the new mask. The sanitizing device can be re-used as many times as needed. The battery can be replaced when it is depleted. Alternatively, a re-chargeable battery can be used instead. While envisioning a lightweight battery that can be connected to the housing and also adhered onto the mask, the invention does not require a particular design with regard to the battery. Therefore, for more professional uses it may be required to have more LEDs and a greater battery supply, in which case the wire can be connected to a larger battery source that can be located or clipped onto, held or stored on the person wearing the mask as well as any person or object in proximity to the mask, such that it is electrically connected to the sanitizing device according to the invention.

In an alternative embodiment, the sanitizing device can be permanently mounted in an aperture in a face mask so that it is not removable, or can be retro-fitted into a valve nozzle in an existing mask. The power source can be in the form of a battery that is attached to the mask, or can be in the form of a conductive material such as graphene, that is printed on the mask itself and connected to the LED assembly through the material of the mask.

While this invention calls for inhalation and exhalation to be the driving force in moving air in and out of the body, the invention is not limited thereby. In one embodiment, a micro fan can be used to assist in certain cases where it may be necessary.

It is believed that this invention will add another layer of protection to masks used for mitigation of disease.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows an exploded view of the front of the sanitizing device according to the invention;

FIG. 2 shows an exploded view of a rear of the sanitizing device according to the invention;

FIG. 3 show one embodiment of the sanitizing device in use on a disposable face mask;

FIG. 4 shows the embodiment of FIG. 3 being worn by a person;

FIG. 5 shows another embodiment of the sanitizing device mounted in another face mask and worn by a person; and

FIG. 6 shows a block dileanagram of the components of the sanitizing device according to the invention.

### DETAILED DESCRIPTION OF THE PREFERRED EMBODIMENT

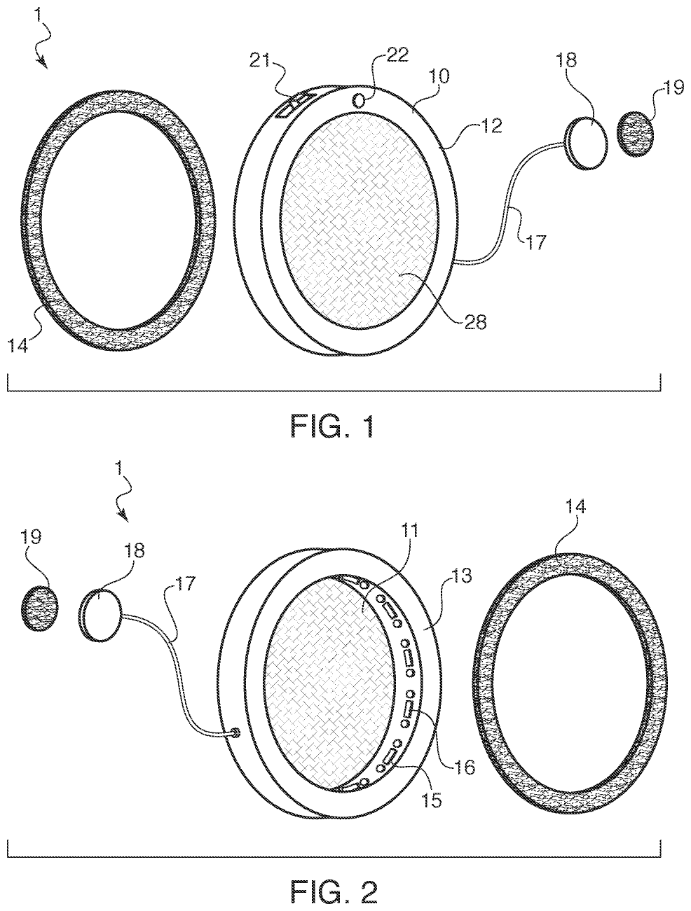

Referring now in detail to the drawings. FIGS. 1 and 2 show an exploded view of the components of the sanitizing device according to the invention. Sanitizing device 1 is formed from a housing 10 having a central aperture 11, overhanging flanges 12, 13 and an attachment layer 14 that is configured to be adhered to the rear flange 13 and attach the housing 11 to a device such as a face mask 20, as shown in FIG. 13. Housing 11 can be made of any suitable lightweight material, such as plastic or aluminum. Inside housing 11 is a UV LED assembly 15, comprised of individual UV LED's 16, connected by a wire 17′ (shown in FIG. 6). UV assembly 15 is recessed behind flanges 12, 13 so that the light emitted therefrom is not directed to the user or to other people facing the user. The number of UV LEDs in the assembly can vary based on the user's preferences, the size of housing and the amount of power that is able to be supplied to the device. A cover layer 28, which can be made of any suitable air-permeable material, is placed over central aperture 11. Also connected to UV LED assembly 15 via wire 17 is a power source 18, which can be in the form of a battery. Power source 18 can also have an attachment layer 19, for attachment to face mask 20. Attachment layers 14, 19, can be formed of adhesive, or preferably a hook-and-loop type closure such as what is known as VELCRO®, which has one part affixed to the housing 10 and the other part affixed to the mask 20, so that the two parts can interlock to attach the housing 10 to the mask 20.

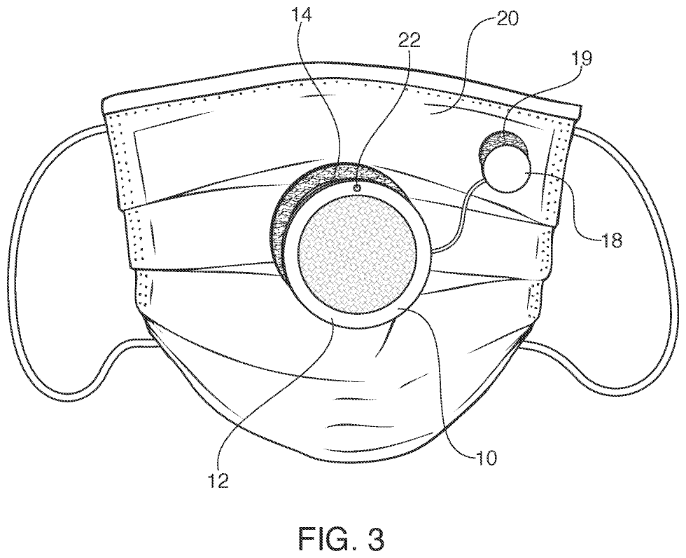

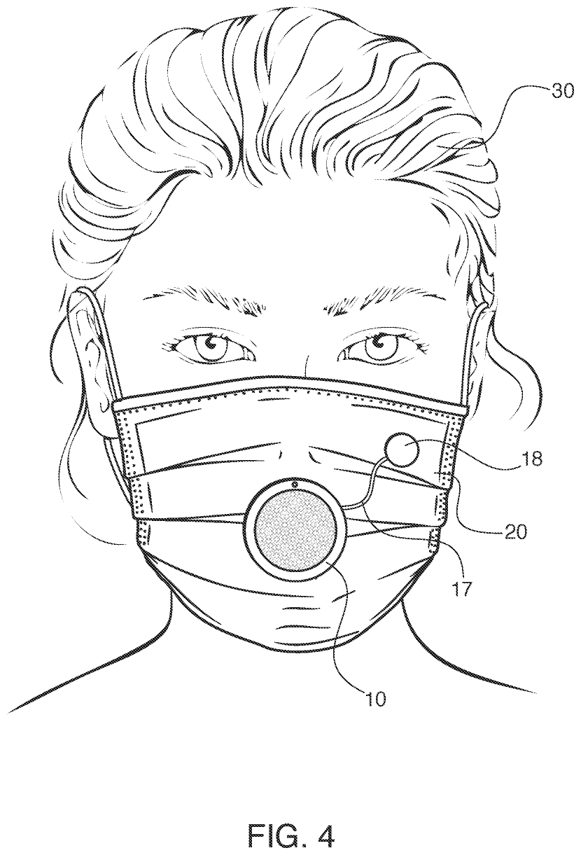

As shown in FIG. 3, housing 10 and power source 18 are attached to the front surface of mask 20, but could also be attached to the rear surface of mask 20. Housing 10 is preferably positioned in the center of mask 20, in front of where a user's 30 mouth would be located, such as shown in FIG. 4. Mask 20 is preferably a disposable surgical mask. Housing 10 and power source 18 can be re-used with different masks, by simply removing attachment layer 14, 19 from the mask when the mask is ready to be discarded, and then applying attachment layers 14, 19 to a new mask. If attachment layers 14, 19 are hook-and-loop type closures, a set of extra closure parts that can be affixed to the mask can be provided, so that a new closure part is applied each time to the new mask.

As shown in FIGS. 1-4, power source 18 is a small flat battery that can be easily applied to face mask 20. However, for other applications, a larger battery can be used, that is not applied to face mask 20 and is instead clipped to the user's belt or other article of clothing. The battery can be disposable or rechargeable.

As shown in the diagram in FIG. 6, switch 21 can be connected to power source 18, to allow the user to turn power on and off to LED assembly 15. In addition, an indicator light 22 can be connected to LED assembly 15, and can be illuminated when power is supplied to LED assembly 15. This is necessary because LED assembly 15 is recessed behind flanges 12, 13 so that light from the LEDs is not easily seen by observers. However, the light from the LED's is projected into central aperture 11 so that any inhalations and exhalation of the user 30 pass through central aperture 11 and are irradiated by UV assembly 15 when the power is on. This helps to decrease the amount of viruses and bacteria that can be transmitted to and from the user via breathing. As shown in the diagram in FIG. 6, a fan can be optionally connected to power source 18, to help move air through central aperture 11.

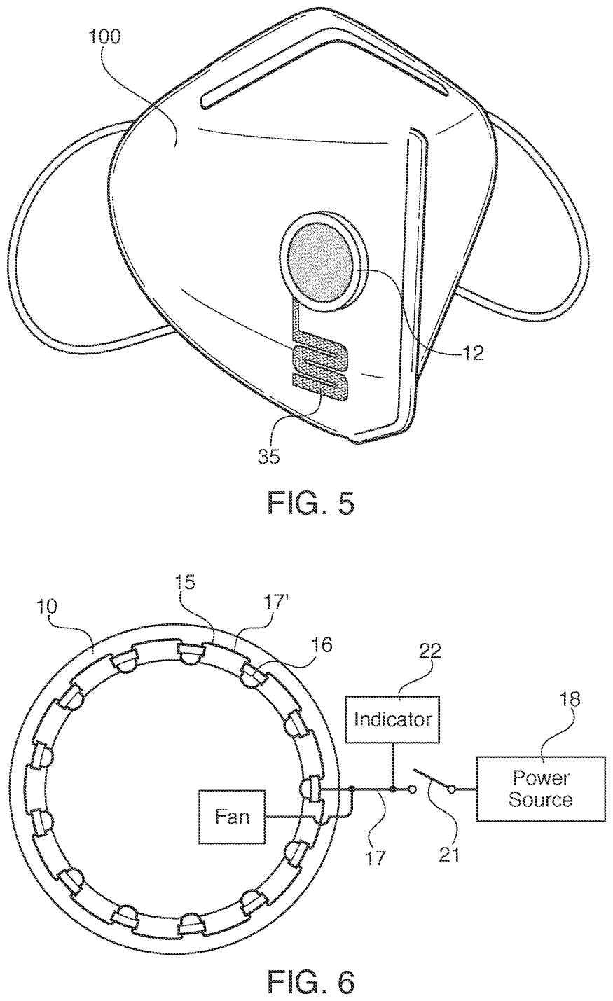

An alternative embodiment of the invention is shown in FIG. 5. Here, housing 10 is mounted in the valve port of a N95 respirator mask 100 make a permanent breathing apparatus. Instead of a removable battery, power source 35 is printed directly on mask 100. Power source 35 can be made of graphene or any other suitable material for supplying power to LED assembly 15. Alternatively, power source 35 could be an auxiliary battery that is clipped to the user's clothing or belt, and connected by a wire. This way, a larger battery could be accommodated in the device according to the invention, and would allow for the use of a greater number or LED's or a longer battery life.

The device of the present invention is a simple and effective way to sanitize the air traveling through a face mask. It is small, lightweight, portable and inexpensive to manufacture.

Accordingly, while only a few embodiments of the present invention have been shown and described, it is obvious that many changes and modifications may be made thereunto without departing from the spirit and scope of the invention.

What is claimed is:

1. A sanitizing device comprising;
a housing having a central aperture,
an ultraviolet (UV) light-emitting diode (LED) assembly mounted in the housing, the UV LED assembly comprising a plurality of UV LEDs arranged circumferentially around the central aperture so as to project UV light into the aperture; and
a power source connected to the UV LED assembly.

2. The sanitizing device according to claim 1, wherein the UV LED assembly is recessed in an interior of the housing and does not extend into the central aperture.

3. The sanitizing device according to claim 1, wherein the power source is a battery.

4. The sanitizing device according to claim 1, further comprising an attachment layer connected to a rear surface of the housing, the attachment layer being configured for attaching the sanitizing device to a face mask.

5. The sanitizing device according to claim 4, wherein the attachment layer is in the form of a hook-and-loop type closure system.

6. The sanitizing device according to claim 5, wherein one side of the hook-and-loop type closure system is fixed to the housing and a corresponding side of the hook-and-loop type closure system is configured to be attached to the face mask, so that the two sides of the hook-and-loop type closure system when placed together connect the device to the mask.

7. The sanitizing device according to claim 3, wherein the battery has an attachment layer on a rear surface thereof, for attaching the battery to a face mask.

8. The sanitizing device according to claim 7, wherein the attachment layer is in the form of a hook-and-loop type closure system.

9. The sanitizing device according to claim 1, further comprising a filter that covers the central aperture on a front side of the housing.

10. The sanitizing device according to claim 1, further comprising a face mask having a front surface and a rear surface and being configured to cover a mouth and nose of a wearer, wherein the sanitizing device and power source are attached to the front or the rear surface of the face mask.

11. The sanitizing device according to claim 10, wherein the power source is a battery.

12. The sanitizing device according to claim 10, wherein the power source is printed on the face mask and is comprised of graphene.

13. The sanitizing device according to claim 10, wherein the housing and power source are connected to the face mask via a hook-and-loop type closure device.

14. The sanitizing device according to claim 10, wherein the housing is permanently affixed in an aperture in the face mask.

15. The sanitizing device according to claim 1, further comprising a fan mounted to the housing and connected to the power source, the fan being configured for moving air through the central aperture.

16. The sanitizing device according to claim 1, further comprising a switch connected to the power source and being configured for turning the power to the UV LED assembly on and off.

17. The sanitizing device according to claim 16, further comprising an indicator light connected to the switch, wherein the indicator light is turned on when power to the UV LED assembly is turned on.
